(19) United States
(12) Patent Application Publication
(10) Pub. No.: US 2025/0338870 A1
(43) Pub. Date: Nov. 6, 2025

(54) METHOD FOR IMPROVING HANDLING PROPERTIES OF PROTEIN INGREDIENTS

(71) Applicant: IMPROVE, DURY (FR)

(72) Inventor: Jean-Charles Jean-Louis, Gerard MOTTE, Jumel (FR)

(21) Appl. No.: 18/720,527

(22) PCT Filed: Dec. 16, 2022
(86) PCT No.: PCT/EP2022/086285

§ 371 Date: Jun. 24, 2024

(30) Foreign Application Priority Data
Dec. 17, 2021 (EP) 21306836.4

(51) Int. Cl. A23J 3/14; C12N 9/50; C12N 9/98

(52) U.S. Cl. CPC A23J 3/14; C12N 9/63; C12N 9/98

(57) ABSTRACT

The present invention relates to protein ingredients, such as protein concentrates and protein isolates, having improved handling properties, in particular exhibiting higher bulk density and flowability, while having a low dustiness compared with their reference on the market. The present invention also relates to compositions comprising said protein ingredients. The present invention further relates to a method for obtaining protein ingredients having improved handling properties, wherein said method comprises a compaction step followed by a milling step and, optionally, a classifying step. The claimed method advantageously does not require any thermal treatment, thereby keeping the native properties of the protein ingredients, while being of a low cost.

### CROSS-REFERENCE TO RELATED APPLICATIONS

This application is the U.S. national phase of International Application No. PCT/EP2022/086285 filed Dec. 16, 2022, which designated the U.S. and claims priority to EP 21306836.4 filed Dec. 17, 2021, the entire contents of each of which are hereby incorporated by reference.

### TECHNICAL FIELD OF THE INVENTION

The present invention relates to protein ingredients, in particular protein concentrates and protein isolates, having improved handling properties.

### TECHNICAL BACKGROUND

Many food ingredients are sold in a dry form, i.e. a powder, which is sometimes called flour. These powders (or flours) are generally formulated and are thus the end-products of the ingredient producer, but the raw material of the end user. For the end user, the chemical composition of a food ingredient is a key parameter, as well as the price, the food safety and the ingredient handling.

Powder handling includes different criteria, which are rarely objectivized, such as density, flowability, dustiness, wettability, dispersibility, etc. However, these criteria are often poorly optimized by the ingredient producer, even so it is a key for the user recipe and usage.

Two main approaches for protein concentration are found in the market: dry processes that produce protein concentrate (i.e. having a protein content lower than 80% on dry matter) and wet processes that produce both protein isolate (i.e. having a protein content greater than or equal to 80% on dry matter) and protein concentrate.

Dry processes consist in the separation of protein bodies having a size lower than 10 μm from starch and/or fibers that are generally (but not necessary) of a coarser size (>10 μm) (Schutyser, M. A. I., & van der Goot, A. J. (2011). The potential of dry fractionation processes for sustainable plant protein production. Trends in Food Science and Technology, 22 (4), 154-164.). The particles thus generated are sorted according to their size, density and/or electrostatic behaviour, for example using technologies such as air classification and/or electrostatic separation. To reach a nice protein separation (meaning a high yield and purity), the material is generally milled at a fine particle size of a few microns: d90<40 μm (meaning that 90% of the grains have a particle size of less than 40 μm). After air classification of this ultrafine flour, a fine fraction rich in protein is collected. This protein fraction is generally very fine, for example with a d90 lower than 30 μm, generally close to 20 μm. However, such extremely fine powders are poorly flowable, have a low density, are poorly wettable and dispersible in water. This behaviour is the consequence of the fine particle size and the chemical composition that favour the electrostatic and capillary bounds between particles.

The only known method for improving the properties of a protein concentrate produced by a dry process is a thermal post treatment of the protein fraction. The process consists in a complex combination of protein concentrate mixing with steam into a continuous or batch reactor. The flour is then cooled on a cooling chamber. The product is at this stage into the form of an heterogeneous agglomerate. The product is then milled into a flour using any type of mill (for example beater, hammer, pin, knife, etc.). The products generated by such processes are marketed as “clean label” products and have sometimes improved flowability. However, this process is expensive in investment and in operational costs and the proteins are denatured by the thermal treatment, which is sometime negative for the application.

Wet processes consist in a separation of the protein by the exploitation of their solubility according to the pH (Boye, J., Zare, F., & Pletch, A. (2010). Pulse proteins: Processing, characterization, functional properties and applications in food and feed. Food Research International, 43 (2), 414-431). pH adjustment, thermal treatments and solid/liquid separation allow producing protein rich creams that are ultimately dried into a powder. The main process for protein drying is mostly but not restricted to spray drying. This type of process is generally monitored to optimise the drying (to obtain a moisture lower than 12%), to avoid product over-heating, clogging and microbe development. Particle's size and shape distribution is thus the consequence of the drying process parameters and the chemical composition of the ingredient. Depending on the machine design, the technical choices and the process parameters, it is possible to generate two typologies of particles: some with a strawberry structure or some with an onion structure having different rheological properties.

In wet processes, the powder rheology is the consequence of many other prioritized specifications. When a spray dryer is optimized on the drying efficiency, the operator generally avoids changing settings in order to avoid major process disturbance or interruption. The aim of the operator of a spray dryer is thus mostly to obtain a product of a constant quality whatever is the feed variability rather than optimizing powder rheology.

Adjusting protein isolate flowability on an existing line is thus mostly done by the addition of flowability aids during the drying or after such as silica fumes. However, theses additives are more and more rejected by the market and alternatives are needed. Thus, protein ingredient produced by wet processes are generally poorly flexible when considering the powder rheology and density.

The last alternative for spray dried protein isolate flowability and density improvement is the addition of post treatment, such as wet agglomeration (Glatt for example). These post treatments consist in a wet agglomeration and drying of the powder and involve heat and water, which is expensive in both investment and energy consumption.

The common point between protein ingredients obtained by a wet or dry process is that the powders are currently poorly optimized in terms of flowability, density, dustiness, wettability and water dispersibility. The powders must be improved, preferably by sustainable post-treatment processes that avoid product thermal denaturation and the use of inadequate additives (for example nanoparticles), since changing the production process is rarely an option.

There is therefore a need of a method for producing protein ingredients having improved handling properties, such as a higher bulk density, an improved flowability and/or a low dustiness.

### DESCRIPTION OF THE INVENTION

The inventor has surprisingly found that the handling properties of a protein ingredient, such as a protein concentrate or a protein isolate, can be efficiently improved by compacting the starting material, in order to increase the particle size, followed by milling the obtained compacted product.

In order to be compacted, the starting material needs to have a suitable moisture content, for example obtained by mixing with water.

The claimed method presents the advantages of being easily carried out, without any heat treatment, thereby avoiding denaturation of the protein ingredient while being of a low cost and, when using a protein concentrate as starting material, without adding any additive.

Combining particle compaction of a protein ingredient in big pellets with their grinding into a relatively coarse particle size allows increasing the particle size of the protein ingredient.

The protein ingredient obtained by the claimed method has advantageously at least one improved handling property, such as an increased bulk density, a decreased dustiness, an improved flowability, an improved dispersibility and/or an improved wettability.

Advantageously, no by-products are generated during the method of the invention, when rejects of the classification (sieving or air classification) are recycled into the mechanical press.

The improvements allowed by the claimed method is key to reduce storage and transport cost, while increasing the applicative value of the protein ingredient by the formulator.

The method of the invention is particularly suitable when using a protein concentrate or a protein isolate as a starting material.

A first object of the invention is a protein ingredient, wherein said protein ingredient is a protein isolate or a protein concentrate, wherein said protein ingredient is in a dry form and wherein said protein ingredient has at least one of the following properties:
said protein ingredient comprises at least 90% of particles having a diameter greater than 100 μm,
said protein ingredient has an aerated density greater than 0.5 g·cm⁻³,
said protein ingredient has a tapped density greater than 0.6 g·cm⁻³,
said protein ingredient has a low convexity, a low elongation and a homogeneous circularity,
said protein ingredient is flowable,
said protein ingredient is dispersible and/or
said protein ingredient is wettable.

The protein ingredient as defined above may be obtained by a process comprising a step of compressing a protein isolate or a protein concentrate followed by a step of milling.

Another object of the invention is a composition comprising at least one protein ingredient as defined above.

The composition as defined above may be a food composition, a feed composition, a pet-food composition, a cosmetic composition, a nutraceutical composition or a pharmaceutical composition.

Another object of the invention is a method for producing a protein ingredient as defined above, wherein said method comprises:
a) providing a protein concentrate or a protein isolate in the form of a wet powder, preferably having a moisture content comprised from 6% to 16%, more preferably from 8% to 14%,
b) compressing the wet powder of step a), to obtain pellets,
c) milling the pellets obtained in step b), to obtain a powder, and
d) classifying (for example sieving or air classifying) the powder obtained in step c), to obtain at least one target coarse fraction, optionally a fine fraction, and optionally a coarse fraction having a size greater than those of the target coarse fraction(s).

In the method as defined above, step b) may comprise compressing both the wet powder of step a) and the fine fraction obtained in step d).

In the method as defined above, step d) may comprise classifying the powder obtained in step c), to obtain a fine fraction, at least one target coarse fraction and a coarse fraction having a size greater than those of the target coarse fraction(s) and step c) may comprise milling both the pellets obtained in step b) and said coarse fraction having a size greater than those of the target coarse fraction(s), to obtain a powder.

The target coarse fraction may be obtained after at least one sieving comprised from 100 μm to 2000 μm.

The method as defined above is preferably carried out in a continuous mode.

The method as defined above preferably comprises:
a) providing a protein concentrate or a protein isolate in the form of a wet powder, preferably having a moisture content comprised from 6% to 16%, more preferably from 8% to 14%,
b) compressing both the wet powder of step a) and the fine fraction obtained in step d), to obtain pellets,
c) milling the pellets obtained in step b) and, optionally, the coarse fraction obtained in step d) which has a size greater than those of the target coarse fraction(s), to obtain a powder, and
d) classifying the powder obtained in step c), to obtain a fine fraction, at least one target coarse fraction, and, optionally, a coarse fraction having a size greater than those of the target coarse fraction(s).

Another object of the invention is the use of compression followed by milling for improving at least one handling property of a protein isolate or a protein concentrate.

Another object of the invention is a system suitable for carrying out the method as defined above, wherein said system comprises:
optionally, at least one mixer,
at least one mechanical press,
at least one mill, and
at least one classification device,
wherein, when present, said mixer is connected to said mechanical press,
wherein said mechanical press is connected to said mill and
wherein said mill is connected to said classification device.

In the system as defined above, said classification device is preferably further connected to said mechanical press and/or to said mill and/or to said mixer.

### Starting Material

The starting material for producing an improved protein ingredient according to the present invention is also a protein ingredient.

A protein ingredient is an ingredient rich in proteins, preferably comprising at least 40% of protein on dry matter, more preferably at least 50% of protein on dry matter.

By the term “protein”, it is particularly meant a biomolecule comprising at least one chain of amino acids residues joined together by peptide bonds. A protein typically comprises at least one chain of at least ten amino acid residues joined together by peptide bonds.

A protein particularly has a molecular weight greater than 1000 Da.

The protein ingredient used as a starting material is preferably provided in the form of a powder.

The protein ingredient as defined above used as a starting material may be obtained from any suitable source, such as a plant, a plant-based material, algae, insects, yeast, fungus or non-human animal (for example milk, fish).

In a preferred embodiment, the protein ingredient as defined above used as a starting material is obtained from a non-animal source.

The plant may be selected from the group consisting of legume, oilseed plant and cereal.

By “legume” is meant herein plants of the Fabaceae family (also called Leguminosae family). The Fabaceae family includes the following subfamilies: Cercidoideae (including the Bauhinia and Cercis genera), Detarioideae (including the Amherstia, Detarium and Tamarindus genera), Duparquetioideae (including the Duparquetia genera), Dialioideae (including the Dialium genera), Caesalpinioideae (including the Caesalpinia, Senna, Mimosa and Acacia genera), and Faboideae (including the Lupinus and Pisum genera).

The plant is preferably a plant of the Faboideae subfamily, more preferably of the Cicer, Glycine, Lathyrus, Lens, Lupinus, Medicago, Phaseolus, Pisum, Trifolium, Vicia, or Vigna genus.

Oil seeds plants include for example sunflower, rapeseed, soy, linseed, canola or camellia.

Cereals for example included wheat, buckwheat, oat, barley, corn or rice.

Any part of the plant may be used, such as shoot, leaf, blossom, needles, stems, branch, fruiting body, fruit, seed, root, corm and/or rhizome.

The protein ingredient as defined above used as a starting material is preferably obtained from plant seeds.

Seeds are preferably used in the form of flour, dehulled seeds or cake.

A preferred plant source is pulse.

By “pulse”, it is herein meant the edible seed from a legume plant.

Preferred pulses include for example pea, yellow pea, chickpea, faba bean, mung bean, lentils, beans, chickpea, lupine, bambara groundnut and/or pongamia.

The plant-based material may be selected from the group consisting of industrial or agricultural by-products, such as malt, bran, brewer spent grain or straw.

The protein ingredient may also by a purified protein composition, such as an enzyme composition.

The enzyme composition used as starting material preferably comprises at least 50% of protein by weight of dry matter, for example at least 75% of protein by weight of dry matter.

The enzyme composition used as starting material preferably comprises at least 50% of protein by weight of the dry matter excluding sugars, for example at least 75% of protein by weight of the dry matter excluding sugars.

Said sugars, in particular maltodextrin and/or maltodextrin analogs, are typically used in enzyme compositions as stabilizing agents.

The enzyme composition may comprise one enzyme or a mix of at least two enzymes.

By the term “enzyme”, it is herein particularly meant a protein, which is a biological catalyst, in particular increasing the rate of a chemical reaction without being changed or consumed in the reaction.

The enzyme may for example by selected from the group consisting of an oxidoreductase, transferase, hydrolase, lyase, isomerase and ligase.

A preferred hydrolase is for example a protease, lipase, amylase or nuclease.

The protein ingredient as defined above is preferably a protein concentrate or a protein isolate.

By “protein concentrate”, it is herein meant a product comprising at least 40% but less than 80% of proteins on dry matter.

A preferred protein concentrate as defined above comprises from 45% to 70% of proteins on dry matter, for example from 50% to 65% of proteins on dry matter.

Most commercialized protein concentrates indeed comprise from 50% to 65% of proteins on dry matter.

Protein concentrates thus still comprise some fibres and carbohydrates.

The protein concentrate used as a starting material may be obtained by any method well known by the skilled person, such as a dry method or a wet method.

An example of dry method for producing a protein concentrate for example comprises:
dehulling and milling the starting material, preferably at a fine particle size of a few microns, to obtain a flour, and
sorting the desired protein concentrate according to their size, density and/or electrostatic behaviour, for example using air classification and/or electrostatic separation.

A wet method for producing a protein concentrate or isolate for example comprises:
dehulling and milling the starting material, preferably at a fine particle size of a few hundred microns, to obtain a flour,
protein solubilising at a neutral or alkaline pH,
a solid/liquid separation,
a protein precipitation in acid or thermal conditions,
a solid/liquid separation to concentrate the precipitated protein,
optionally, a thermal treatment for protein stabilizing and
the protein fraction drying, for example in a spray dryer.

In wet processes, obtaining a protein concentrate or a protein isolate is the consequence of the raw material, the process steps and the process parameters applied.

By “protein isolate”, it is herein meant a product comprising at least 80% of proteins on dry matter, preferably at least 85% of proteins on dry matter.

In a preferred embodiment, the protein isolate comprises from 80% to 95% of proteins on dry matter, more preferably from 85% to 95% of proteins on dry matter.

Contrary to a protein concentrate, a protein isolate does not comprise dietary fibres and starches.

The protein isolate used as a starting material may be obtained by any method well known by the skilled person, such as those defined above.

The method for producing a protein isolate may comprise, starting from flour:
a step of alkaline or neutral extraction followed by a step of acidic precipitation,
optionally, a neutralization step,
optionally, a step of heat treatment, and
a step of drying.

The protein isolate may also be obtained according to the method disclosed in application PCT/EP2021/067468, which method comprises the following steps, starting from flour or dehulled seeds:
a first washing step in acidic conditions, which is optionally followed by a rinse step with an aqueous solution,
a second washing step in an alkaline solution,
optionally, a neutralization step,
optionally, a step of heat treatment, and
a step of drying.

The protein ingredient as defined above may comprise 2% to 15% of lipids, preferably 2% to 6%, by weight of dry matter and/or 2% to 12%, preferably 2% to 8%, of ashes by weight.

By the term “lipid”, it is herein meant a fatty or waxy organic compound that is readily soluble in a nonpolar solvent, but not in a polar solvent. Examples of lipid include wax, oil, sterol, cholesterol, fat-soluble vitamin, monoglyceride, diglyceride, triglyceride (also called fats) or phospholipid.

By “ashes”, it is herein meant the inorganic non-combustible material. Ashes comprise mainly salty inorganic constituents, such as metal salts and minerals.

In one preferred embodiment, the protein ingredient as defined above is a non-animal protein ingredient.

The protein ingredient as defined above is preferably a plant protein ingredient, i.e. obtained from a plant as defined above, still more preferably from a plant seed as defined above.

A preferred protein ingredient used as starting material is a pulse protein concentrate or a pulse protein isolate.

The handling properties of the protein ingredient as defined above can be advantageously improved by carrying out the method according to the invention disclosed below.

### Method for Producing an Improved Protein Ingredient

The present invention thus relates to a method for producing a protein ingredient having at least one improved handling property.

The method as defined above is firstly based on increasing the particle size of the protein ingredient whose handling properties are to be improved, which is achieved by a compressing step, preferably a mechanical compressing step.

If needed, a moisture adjustment step is performed before said compressing step, in order to increase particle cohesion, while increasing the mass balance.

Said compressing step is followed by a milling step, in order to obtain a powder.

The method as defined above preferably further comprises a classifying step, to obtain at least one coarse fraction of the desired particle size, in particular depending on the sieving retained.

The method as defined above can still be improved by recycling to the mechanical press and/or to the mixer the fine fraction having a particle size below those of the target coarse fraction. Recycling the fine fraction obtained after milling advantageously avoid any losses and also ease the mechanical compressing by a precompaction.

The method as defined above may further comprise recycling to the milling, a fraction having a particle size higher than those of the target coarse fraction(s). The recycling of these too coarse particles into the mill allows using a “gentle” milling, thereby providing a better control on the particle size distribution of the target coarse fraction.

The method of the invention is thus preferably carried out in a continuous mode.

By “continuous mode”, it is herein meant that the incoming flow of starting material (i.e. the protein isolate or the protein concentrate) to the system as well as the protein ingredient output is constant during the production. Each step of the method is thus run concurrently with every other step.

The method for producing a protein ingredient as defined above preferably comprises:
a) providing a protein concentrate or a protein isolate in the form of a wet powder,
b) compressing the wet powder of step a), to obtain pellets,
c) milling the pellets obtained in step b), to obtain a powder,
d) classifying the powder obtained in step c), to obtain at least one target coarse fraction, optionally a fine fraction, and optionally a coarse fraction having a size greater than those of the target coarse fraction(s),
e) optionally, compressing the fine fraction obtained in step d) into pellets and milling said pellets, to obtain a powder.

### Step a)

Step a) comprises providing a protein concentrate or a protein isolate in the form of a wet powder.

The wet powder as defined above preferably has a moisture content comprised from 4% to 16%, more preferably from 8% to 14%.

By the expression “moisture content of x%”, it is herein meant that the wet powder comprises x g of water for 100 g of wet powder.

The protein concentrate or protein isolate in the form of a wet powder may for example be obtained by mixing said protein concentrate or protein isolate with water.

Step a) thus for example comprises mixing a protein concentrate or a protein isolate, which protein concentrate or protein isolate is in the form of a powder, with water, to obtain a wet powder.

The protein concentrate and the protein isolate are particularly as defined above in the section “Starting material”.

The protein concentrate is preferably a pulse protein concentrate.

The protein isolate is preferably a pulse protein isolate.

The pulse is preferably selected from the group consisting of pea, soy, faba bean, lentils, beans, chickpea, lupine, yellow pea and mung bean.

As explained above, the method efficiency is particularly high thanks to the moisture content adjustment that increases the particles cohesion will increasing the mass balance of the starting material.

The moisture adjustment can be performed in any type of suitable mixer.

The mixer is preferably a continuous mixer.

Mixing the protein concentrate or protein isolate with water is preferably carried at ambient temperature, for example from 10° C. to 40° C., preferably from 15° C. to 35° C., more preferably from 18° C. to 25° C.

Water is preferably pure or drinkable water, in particular when the protein ingredient is to be used in food or feed compositions.

In one embodiment, step a) may comprise mixing the protein concentrate or protein isolate with water and with the fine fraction obtained in step d).

The method as defined above preferably does not comprise adding any additive.

In particular, step a) preferably does not comprise mixing the protein concentrate, the protein isolate or the wet powder with any additive.

However, when the protein ingredient is a protein isolate, step a) may comprise mixing the protein isolate with water and one or at least one additive, preferably chosen among a pregelatinized starch or a lipid source (for example oil, seeds oil).

### Step b)

Step b) comprises compressing the wet powder of step a), to obtain pellets.

Step b) preferably comprises mechanical compressing the wet powder of step a), to obtain pellets.

Step b) may be carried out in a mechanical press.

Any suitable mechanical press may be used, such as tangential roll presses produced by Euragglo (for example K.R. KOMAREK type B220B). Such presses consist in a powder pre-compaction by a feeding screw that simultaneously transports the product through the two compressing rolls that will do the compaction. These two rolls are profiled with different shapes that creates the pellets. The pressure applied is for example of 35 kN/cm.

By “pellet”, it is herein meant a relatively big, rounded or cigar or tube-shaped, compressed mass of a substance.

The pellet preferably has a size of few centimeters in length and around 1 or 2 centimeter in width.

For example, the pellets may have a length of 10 cm and a diameter of 1 cm.

Step b) may comprise compressing both the wet powder of step a) and the fine fraction obtained in step d).

Step b) is preferably carried out without any thermal treatment and/or any use of additives.

Compressing is particularly carried out at a temperature lower than 50° C.

Compressing is thus preferably an ambient temperature compressing.

The ambient temperature may for example from 10° C. to 40° C., preferably from 15° C. to 35° C., more preferably from 18° C. to 25° C.

### Step c)

Step c) comprises milling the pellets obtained in step b), to obtain a powder.

Step c) may be performed in a mill.

Any suitable mill may be used.

The mill is preferably combined with a classification device, such as sifter or a gravity classifier, for implementing the classifying step d).

Step c) may comprise milling both the pellets obtained in step b) and the coarse fraction having a size greater than those of the target coarse fraction(s) obtained in step d), to obtain a powder.

### Steps d)

Step d) comprises classifying the powder obtained in step c), to obtain at least one target coarse fraction, optionally a fine fraction, and optionally a coarse fraction having a size greater than those of the target coarse fraction(s).

The protein ingredient produced by the method of the invention corresponds to the target coarse fraction(s) obtained in step d).

Classifying may be sieving and/or air classifying.

Classifying may be carried out in a sifter and/or a gravity classifier.

As an example, the target coarse fraction may be obtained after at least one sieving comprised from 100 μm to 2000 μm.

The target coarse fraction may be for example be obtained after at least one sieving comprised from 100 μm to 400 μm or after at least one sieving comprised from 400 μm to 2000 μm.

Using a sieving comprised from 100 μm to 400 μm particularly allows producing a flour.

Using a sieving comprised from 400 μm to 2000 μm particularly allows producing a semolina.

In a preferred embodiment, step d) may comprise classifying the powder obtained in step c), to obtain a fine fraction, at least one target coarse fraction and a coarse fraction having a size greater than those of the target coarse fraction(s) and step c) comprises milling both the pellets obtained in step b) and said coarse fraction having a size greater than those of the target coarse fraction(s), to obtain a powder.

### Optional Step e)

Optional step e) comprises compressing the fine fraction obtained in step d) into pellets and milling said pellets, to obtain a powder.

When the method is run in a continuous mode, instead of step e), step b) comprises compressing both the wet powder of step a) and the fine fraction obtained in step d), to obtain pellets.

The fine fraction obtained in step d) comprises too fine particles, in particular particles of a diameter lower than 100 μm.

The fine fraction is advantageously sent back to the press, in order to ease the compression and avoid any by-product generation.

This recycling may also improve pellet formation and strength.

Optionally, the fine fraction is sent back to the mixer in step a), in order to be mixed with the protein concentrate or isolate and water.

In a preferred embodiment, the method as defined above is carried out in a continuous mode and comprises:
a) optionally, mixing a protein concentrate or a protein isolate with water, to obtain a wet powder, preferably having a moisture content comprised from 4% to 16%, more preferably from 8% to 14%,
b) compressing (i) a protein concentrate or a protein isolate provided in the form of a wet powder or the wet powder of step a) and (ii) optionally, the fine fraction obtained in step d), to obtain pellets,
c) milling the pellets obtained in step b) and, optionally, the coarse fraction obtained in step d) which has a size greater than those of the target coarse fraction(s), to obtain a powder,
d) classifying the powder obtained in step c), to obtain a fine fraction, at least one target coarse fraction, and, optionally, a coarse fraction having a size greater than those of the target coarse fraction(s).

The protein ingredient obtained by the method defined above advantageously has (i) an increased density and (ii) preferably, an improved flowability, an improved wettability, an improved dispersibility and/or a decreased dustiness.

The method as defined above has a high interest for improving protein concentrates produced by dry processes, in particular which consist in a micronization and an air classification. As explained above, such dry process generates particles that are mandatory of very fine size (d50<30 μm), which is highly negative in terms of powder handling.

The method as defined above can be implemented directly after the ingredient production line (i.e. the protein concentrate or isolate production line) as a post treatment or before the formulation, for example to ease the handling before mixing, extrusion, etc.

The method as defined above may thus comprise or not, before step a), the steps of a method for producing a protein concentrate or a protein isolate.

The method as defined above may thus comprise or not, after step d) or e), the steps of a method for formulating the protein ingredient.

The method as defined above may be carried out in a system as disclosed below.

The method as defined above does not require any drying step, which is particularly advantageous since drying is energy consuming.

The method as defined above thus particularly does not comprise any drying step.

### System for Producing a Protein Ingredient

The present invention also relates to a system for producing a protein ingredient having at least one improved handling property as defined below, wherein said system comprises:
optionally, at least one mixer,
at least one mechanical press,
at least one mill, and
at least one classification device.

Said system is suitable for carrying out the method as defined above for producing an improved protein ingredient.

The system as defined above is preferably characterized in that:
when present, said mixer is connected to the mechanical press, in particular so as the wet powder produced in the mixer is introduced into the mechanical press,
said mechanical press is connected to the mill, in particular so as the pellets produced by the mechanical press are introduced into the mill and
said mill is connected to said classification device, in particular so as the powder produced by the mill is introduced into the classification device.

When the system as defined above comprises more than one mixer, more than one mechanical press, more than one mill and/or more than one classification device, at least one mixer is connected to at least one mechanical press, at least one mechanical press is connected to at least one mill and at least one mill is connected to at least one classification device. The devices having the same function may be mounted in series or in parallel.

In a preferred embodiment, said classification device is connected to said mechanical press, in particular so as the fine fraction produced by the classification device is introduced into the mechanical press and/or is connected to said mill, in particular so as the coarse fraction having a size greater than those of the target coarse fraction(s) produced by the classification device is introduced into the mill.

In still a preferred embodiment, said clarification device is connected both to said mechanical press and to said mill.

The mixer as defined above particularly comprises:
means for mixing,
at least one inlet suitable for introducing the starting material, in particular the protein isolate or the protein concentrate,
at least one inlet suitable for introducing water,
optionally, at least one inlet suitable for introducing food grade additive(s),
optionally, at least one inlet suitable for introducing a fine fraction, which inlet is connected to the outlet for the fine fraction of the classification device, and
at least one outlet for the wet powder, which outlet is connected to a mechanical press inlet, for example by gravity or any compatible mechanical or air conveying.

The mixer as defined above is preferentially a continuous mixer.

The mechanical press particularly comprises:
at least one inlet suitable for introducing the wet powder, which inlet is connected to the outlet for the wet powder of the mixer.
optionally, at least one inlet suitable for introducing a fine fraction, which inlet is connected to the outlet for the fine fraction of the classification device,
means for compressing, such as profiled rolls (cigar shapes, spheres or ball shape for example) or perforated wheels, and
at least one outlet for the pellets, which outlet is connected to the inlet suitable for introducing the pellets of the mill.

The mill as defined above particularly comprises:
means for milling,
at least one inlet suitable for introducing the pellets, which is connected to the outlet for the pellets of the mechanical press,
at least one outlet for the powder, which outlet is connected to the inlet suitable for introducing the powder of the classification device,
optionally, at least one screen, and
optionally, at least one inlet suitable for introducing a coarse fraction having a size greater than those of the target coarse fraction(s), which inlet is connected to the outlet for the coarse fraction having a size greater than those of the target coarse fraction(s) of the classification device.

The mill is ideally operating at a very low rotation speed to avoid to many fine particles generation.

The classification device as defined above particularly comprises:
means for particle separation according to their size, such as at least one sieve or air classification system,
at least one inlet suitable for introducing the powder, which is connected to the outlet for the powder of the mill,
at least one outlet for the or each target coarse fraction,
optionally, at least one outlet for the fine fraction, which is connected to the inlet suitable for introducing the fine fraction of the mechanical press or of the mixer, and
optionally, at least one outlet for the coarse fraction having a size greater than those of the target coarse fraction, which outlet is connected to the inlet suitable for introducing said coarse fraction in the mill.

The classification device may for a sifter or a gravity classifier.

The system may comprise both at least one sifter and at least one gravity classifier.

In such system, the mill is preferably connected to the sifter and said sifter is connected to the gravity classifier.

The system as defined above is for example a production plant.

### Improved Protein Ingredient

The present invention thus also relates to an improved protein ingredient, i.e. a protein ingredient having at least one improved handling property, in particular by comparison to protein ingredients obtained by standard methods, such as the protein ingredients defined in the section “starting material”, which are used as starting material in the method of the invention.

The present invention particularly relates to a protein ingredient, wherein said protein ingredient is a protein isolate or a protein concentrate, wherein said protein ingredient is in a dry form and wherein said protein ingredient has at least one improved handling property.

The terms “protein isolate”, “protein concentrate” and “dry form” are particularly as defined above in the section “starting material”.

The protein ingredient is preferably a non-animal protein ingredient.

The protein ingredient is preferably a plant protein ingredient or an enzyme composition.

Since all the steps of the method of the invention are carried out at temperatures lower than 50° C., in particular at ambient temperature, the method according to the invention advantageously does not denature the proteins of the protein ingredient.

Thus, when the protein ingredient is an enzyme composition, the enzymatic activity of the improved protein ingredient is preferably substantially the same as those of the starting material. The enzymatic activity of the improved ingredient is preferably equal to at least 90%, more preferably at least 95% of the enzymatic activity in the starting material, i.e. in the protein ingredient before carrying out the method of the invention.

The improved protein ingredient as defined above is preferably obtained by the method disclosed above, in particular comprising a step of compressing a protein isolate or a protein concentrate followed by a step of milling and, optionally, a step of classifying (for example by sieving and/or air classifying).

The friability of the improved protein ingredient is preferably lower than those of the protein ingredient before carrying out the method of the invention. This criterion can be assessed by any compatible laboratory device, such as friability tester (for example FT2 from HTDS® or homemade analogue devices) coupled with particle size distribution (Malvern®) or Dustmeter (Heubach®). The concept of these tests is to evaluate the proportion of dust generated after a specific and standard manipulation.

The improved protein ingredient as defined above is a protein isolate or a protein concentrate and is in a dry form.

By the expression “dry form”, it is herein meant that the improved protein ingredient is in the form of a powder having a moisture content below 16%.

The handling property may be selected from the group consisting of density, flowability, dustiness, dispersibility and wettability.

By “density”, it is herein meant the mass of a unit volume of a product. It can be expressed in both bulk density (native density) or tapped density (after vibration). Density reflects the degree of compactness of a product.

Increasing the density of a protein ingredient advantageously allows reducing the volume of storage of said product.

Density may be aerated density or tapped density.

By “aerated density”, it is herein meant the volume occupied by a known mass of product after a free filling of the volume to have the standard arrangement degree between particles (for example ISO 11272:2017 or ISO 697:1981). Aerated density is sometimes called bulk density. Aerated density may be measured by any method well known by the skilled person, such as a standard test tube or any vessel calibrated in volume after introducing inside a known mass of samples without any further product manipulation.

Aerated density is for example measured in a density analyzer (for example Autotap, Quantchrome®).

By “tapped density”, it is herein meant the volume occupied by a known mass of product after vibration of the product in order to have the maximal arrangement degree between particles (for example ISO 11272:2017 or ISO 697:1981). Tapped density may be measured by any method well known by the skilled person, such as a standard test tube or any vessel calibrated in volume after introducing inside a known mass of samples and after standard and repetitive vibration of the product assisted or not by a laboratory device (powder rheometer or autotap system for example).

Tapped density is for example measured in a density analyzer (for example, Autotap, Quantchrome®).

By “flowability”, it is herein meant the capacity of a product to move by flow, without sticking.

Flowability may be measured by any method well known by the skilled person, such as Hausner index, which is the ratio of tapped to aerated density itself determined by relevant methods (for example ISO 11272:2017 or ISO 697:1981). The Hausner index is sometime express as Carr index, which is the reverse of the Hausner index.

A product is flowable if its Hausner index is lower than 1.2.

Flowability is preferably measured by the Hausner index.

By “dustiness”, it is herein meant the tendency of particles to become airborne.

Dustiness may be measured by any method well known by the skilled person, such as a particle size distribution, for example using a particle size analyzer (for example Malvern®) or the use of a dustmeter (for example Heubach®).

A product has a low dustiness if the proportion of particles below 20 μm is low.

For example, a product having at least 90% of particles with a diameter greater than 50 μm has a low dustiness.

The expression “at least x% of particles with a diameter greater than y μm” particularly means that the volume occupied by the particles having a diameter greater than y μm is equal to at least x% of the total volume occupied by the particles.

Diameter of the particles is for example measured in a particle size analyzer in particular by laser diffraction in dry mode (for example, Mastersizer 3000, Malvern®). The distribution parameter is preferably the median and the distribution is preferably a volumetric distribution.

By “wettability”, it is herein meant the degree to which a solid is wetted by a liquid, measured by the force of adhesion between the solid and liquid phases.

Wettability may be measured by any method well known by the skilled person, such as the one disclosed in the following article: Felix da Silva, D., Tziouri, D., Ahrné, L., Bovet, N., Larsen, F. H., Ipsen, R., & Hougaard, A. B. (2020). Reconstitution behavior of cheese powders: Effects of cheese age and dairy ingredients on wettability, dispersibility and total rehydration. Journal of Food Engineering, 270 (September 2019). https://doi.org/10.1016/j.jfoodeng.2019.109763.

Wettability is for example measured by dispersing 5 g of the powder sample, in particular of the protein ingredient, on a surface of 100 mL of static tap water inside a beaker and measuring the time required for the powder to sink into the water (i.e. to be wet), for example using a camera.

A product is considered as well wettable if the time for the powder to be wet is below 10 minutes. Above 60 minutes, the powder is poorly wettable.

By “dispersibility”, it is herein meant the ability to be spatially well distributed in terms of size and concentration in a liquid. Dispersibility may be measured by any method well known by the skilled person, such as the one disclosed in the following article: Felix da Silva, D., Tziouri, D., Ahrné, L., Bovet, N., Larsen, F. H., Ipsen, R., & Hougaard, A. B. (2020). Reconstitution behavior of cheese powders: Effects of cheese age and dairy ingredients on wettability, dispersibility and total rehydration. Journal of Food Engineering, 270 (September 2019). https://doi.org/10.1016/j.jfoodeng.2019.109763.

A product is dispersible if more than 50% by weight of the product can be solubilized.

Dispersibility is for example measured by powder dispersion in a 5% water solution (w/w), followed by stirring, for example during 6 minutes, filtering the obtained solution and measuring the weight of solid present in the filter.

Improved handling properties of the protein ingredient are for example (i) a higher density and (ii), preferably, a higher flowability, a lower dustiness, a higher wettability and/or a dispersibility, in particular by comparison to protein ingredients obtained by standard methods, such as the protein ingredients defined above in the section “starting material” which are used as starting material in the method of the invention.

The improved protein ingredient for example has at least one, preferably at least two, at least three, at least four or the five following handling properties:
said protein ingredient has a higher density, in particular by comparison to protein ingredients obtained by standard methods, such as the protein ingredients defined above in the section “starting material”,
said protein ingredient has a low dustiness,
said protein ingredient is flowable,
said protein ingredient is wettable and/or
said protein ingredient is dispersible.

The improved protein ingredient as defined above has also a specific shape, characterized by a low convexity, a low elongation and an homogeneous circularity.

Convexity, elongation and circularity may be measured by image analysis, for example using a particle shape analyzer in dry mode (such as Morphology G3, Malvern®).

By “convexity”, it is herein meant the presence of convex angles within the particles indicating a tortuous shape. The classical range is from 0.5 to 1 with an exponential behavior. The value “1” corresponds to particles without any concave angles (as a sphere but also a triangle for example).

A low convexity for example means that at least 90% of the particles have a value lower than 0.98, in particular as measured in a particle shape analyzer in dry mode (Morphology G3, Malvern®).

By “elongation”, it is herein meant an elongated shape or not of the particles. The possible range is from 0 (not elongated) to 1 (infinitely elongated) with an exponential behavior. A value of 0.5 indicates that the particles are not very elongated since their length is generally less than 2 times their width.

A poor elongation, also called low elongation, for example means that at least 90% of the particles have a value lower than 0.5, as measured in a particle shape analyzer in dry mode (such as Morphology G3, Malvern®).

By “homogeneous circularity”, it is herein meant the absence of angles within the particles. By the criteria “HS circularity”, the possible range is from 0 to 1. The value “1” correspond to perfect circular particles while 0 corresponds to an infinitely angular shape.

An homogeneous circularity for example means that at least 10% of the particles have a value greater or equal to 0.40 with a span lower than 0.6, as measured in a particle shape analyzer in dry mode (such as Morphology G3, Malvern®).

The improved protein ingredient as defined above preferably has at least one, at least two, at least three, at least four, at least five or at least six of the following properties or the seven following properties:
said protein ingredient comprises at least 90% of particles having a diameter greater than 100 μm, preferably greater than 150 μm, more preferably greater than 200 μm, still more preferably greater than 250 μm,
said protein ingredient has an aerated density greater than 0.5 g·cm⁻³, preferably greater than 0.6 g·cm⁻³,
said protein ingredient has a tapped density greater than 0.6 g·cm⁻³, preferably greater than 0.7 g·cm⁻³,
said protein ingredient has a low convexity, a low elongation and a homogeneous circularity,
said protein ingredient is flowable,
said protein ingredient is dispersible and/or
said protein ingredient is wettable.

The improved protein ingredient as defined above may further comprise:
at least 10% of particles having a diameter greater than 15 μm, preferably greater than 20 μm, and/or
at least 50% of particles having a diameter greater than 30 μm, preferably greater than 50 μm, more preferably greater than 100 μm, still more preferably greater than 150 μm.

In another preferred embodiment, the improved protein ingredient as defined above is a protein concentrate, is in a dry form, and has at least two, at least three, at least four, at least five of or the six following properties:
said protein ingredient comprises at least 90% of particles having a diameter greater than 400 μm, preferably greater than 450 μm, more preferably greater than 500 μm,
said protein ingredient has an aerated density greater than 0.5 g·cm⁻³, preferably greater than 0.6 g·cm⁻³,
said protein ingredient has a tapped density greater than 0.6 g·cm⁻³, preferably greater than 0.7 g·cm⁻³,
said protein ingredient has a low convexity, a low elongation and a homogeneous circularity,
said protein ingredient is flowable and/or
said protein ingredient is dispersible.

In another preferred embodiment, the improved protein ingredient as defined above is a protein concentrate, is in a dry form, and has at least two, at least three of or the four following properties:
said protein ingredient comprises at least 90% of particles having a diameter greater than 200 μm, preferably greater than 250 μm, more preferably greater than 300 μm, and lower than 400 μm,
said protein ingredient has an aerated density greater than 0.5 g·cm⁻³, preferably greater than 0.6 g·cm⁻³,
said protein ingredient has a tapped density greater than 0.6 g·cm⁻³, preferably greater than 0.7 g·cm⁻³,
said protein ingredient has a low convexity, a low elongation and a homogeneous circularity.

In another preferred embodiment, the improved protein ingredient as defined above is a protein isolate, is in a dry form and has at least one, two or the three following properties:
said protein ingredient comprises at least 90% of particles having a diameter greater than 150 μm, preferably greater than 200 μm, more preferably greater than 250 μm,
said protein ingredient has an aerated density greater than 0.5 g·cm⁻³,
said protein ingredient is flowable.

### Composition Comprising an Improved Protein Ingredient

The present invention also relates to a composition comprising at least one protein ingredient as defined above in the section “Improved protein ingredient”.

The composition as defined above may comprise from 0.01% to 99% of protein ingredient, the percentage being expressed by weight of the composition.

The composition as defined above may for example comprise from 0.01% to 5% of protein ingredient, preferably from 0.1% to 5% of protein ingredient, more preferably from 1% to 5% of protein ingredient, the percentage being expressed by weight of the composition.

Alternatively, the composition as defined above may comprise from 5% to 90% of the protein ingredient, preferably from 5% to 80% of the protein ingredient, more preferably from 5% to 70% of the protein ingredient, more preferably from 5% to 60% of the protein ingredient, still more preferably from 5% to 50% of the protein ingredient, for example from 5% to 40% of the protein ingredient or from 5% to 30% of the protein ingredient, the percentage being expressed by weight of the composition.

If at least two protein ingredients as defined above are used in the composition, the above percentages correspond to the total weight of the protein ingredients of the invention.

The composition as defined above is preferably a food composition, a feed composition (such as but not limited to aquafeed), a pet-food composition, a cosmetic composition, a nutraceutical composition or a pharmaceutical composition.

The food composition, in particular for human consumption, may for example comprise or consist of a meat substitute or analogue, a dairy substitute or analogue, a snack, a cereal product, or a functional product suitable for sport nutrition. The food composition may for example comprise or consist of a plant-based yoghurt, plant based beverage or high protein snack.

### Use of Compression and Milling for Improving Handling Properties

The present invention also relates to the use of at least one mechanical press, at least one mill and, optionally at least one classification device, for improving at least one handling property of a protein ingredient, preferably of an isolate or a protein concentrate, preferably in a continuous mode.

The present invention also relates to the use of compression followed by milling and, optionally, classifying (for example sieving and/or air classifying) for improving at least one handling property of a protein ingredient, preferably of an isolate or a protein concentrate, preferably in a continuous mode.

The isolate or protein concentrate is for example as defined above in the section “starting material”.

The handling property, mechanical press, mill, classification device, compression and milling are for example as defined above.

The handling property may for example be selected for the group consisting of density, flowability, dustiness, wettability and/or a dispersibility.

The present invention will be further illustrated by the FIGURE and examples below.

### BRIEF DESCRIPTION OF THE DRAWINGS

FIG. 1 is a general scheme of a process for obtaining an improved protein ingredient according to the invention using a protein isolate or a protein concentrate as starting material.

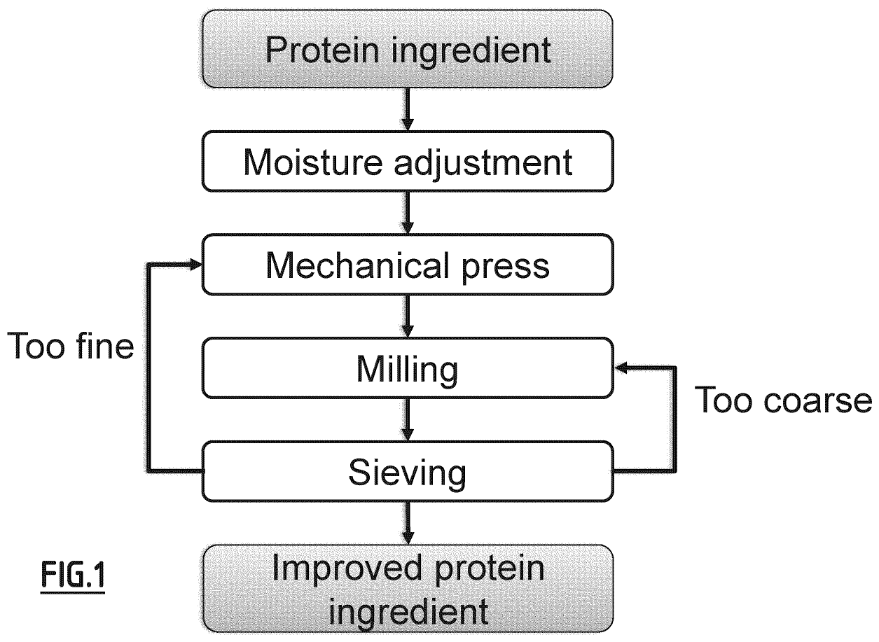

### EXAMPLES

### Example 1: Improving a Protein Concentrate by Compressing and Roller Milling

A standard pea protein concentrate produced by Vestkorn have been tested on a roll press KR KOMAREK B220 B (Euragglo). In a native state, the pea proteins are not successfully compacted since the particle cohesivity is initially too low. The initial aerated density was of 0.41 g·cm⁻³ and the initial tapped density was of 0.53 g·cm⁻³. A moisture adjustment has been done by adding 4% of water. Thanks to this initialisation, pellets are produced without needs of thermal treatment. Pellets are then milled on a two-stage roller mill process and sieved between 100 and 315 μm. The fraction bellow 100 μm is send back to the mechanical press in order to obtain a better compaction by an increase of density into the compaction shapes.

Two types of powder can be generated by this example:
1) A protein concentrate having a “flour” texture, for example obtained after a sieving between 100 and 315 μm, and
2) A protein concentrate having a semolina texture, for example obtained after a sieving at 315 μm.

The following Table 1 shows the main characteristics of the ingredients thus improved.

TABLE 1

| | | | Improved pea | | Improved pea |
|---|---|---|---|---|---|
| | Native pea | | protein | | protein |
| | protein | | concentrate | | concentrate |
| | concentrate | | “flour” | | “semolina” |
| Aerated density | 0.41 | | 0.64 | | 0.69 |
| (g · cm−3) | | | | | |
| Tapped density | 0.53 | | 0.78 | | 0.75 |
| (g · cm−3) | | | | | |
| Hausner index | 1.28 | | 1.20 | | 1.10 |
| Wettability (min) | 18 | min | 60 | min | 1 min |
| Dispersibility | >6 | min | >6 | min | 59% |

TABLE 2

| Parameter | Sample Name | d10 (v) | d50 (v) | d90 (v) | Span (v) |
|---|---|---|---|---|---|
| Diameter (μm) | Native | 7.2 | 15.1 | 33.0 | 1.7 |
| | Flour | 23.1 | 206.6 | 329.3 | 1.5 |
| | Semolina | 272.6 | 429.6 | 550.9 | 0.6 |
| Convexity | Native | 0.85 | 0.95 | 0.99 | 0.15 |
| | Flour | 0.83 | 0.89 | 0.97 | 0.15 |
| | Semolina | 0.83 | 0.86 | 0.90 | 0.09 |
| Elongation | Native | 0.14 | 0.34 | 0.55 | 1.22 |
| | Flour | 0.10 | 0.32 | 0.48 | 1.19 |
| | Semolina | 0.15 | 0.27 | 0.39 | 0.88 |
| HS Circularity | Native | 0.39 | 0.64 | 0.86 | 0.74 |
| | Flour | 0.49 | 0.64 | 0.80 | 0.48 |
| | Semolina | 0.49 | 0.59 | 0.69 | 0.35 |

The improved protein ingredient samples (flour and semolina) are characterized by a low convexity (d90<0.98), a poor elongation d90<0.5 but a higher and more homogeneous circularity (d10>=0.40 and Span<0.6) (see Table 2).

### Example 2: Improving a Protein Isolate by Compressing and Roller Milling

The method of the invention has been carried out on a commercial pea isolate as starting material.

The features of the improved pea isolate by comparison to the starting material are shown in Table 3.

TABLE 3

| | | | | Diameter | Diameter | Diameter |
|---|---|---|---|---|---|---|
| | Aerated | Tapped | | in μm | in μm | in μm |
| | density | density | Hausner | D10 | D50 | D90 |
| Pea isolate | 0.50 | 0.67 | 1.32 | 39.7 | 72.8 | 123.3 |
| (starting material) | | | | | | |
| Improved pea | 0.52 | 0.56 | 1.15 | 85.5 | 160.2 | 269.1 |
| isolate | | | | | | |

The Hausner index shows that fluidity is improved by the claimed method, since the improved pea isolate is fluid, contrary to the starting material.

### Example 3: Improving an Enzyme Composition by Compressing and Roller Milling

The method of the invention has been carried out on several commercial powder enzymes as starting material.

All of them showed a high compaction ability through the formation of strong pellets. Subsequently, the aerated density was significantly increased of at least 10%. No significant reduction of the enzymatic activity was observed before and after treatment (see Table 4).

TABLE 4

| | | | Enzymatic |
|---|---|---|---|
| | | Ability for | activity |
| Commercial name | Enzyme type | compaction | reduction |
| Papaine performase | Neutral protease | High | <5% |
| PNS 1000 | | | |
| Prolyve BS CONC | Neutral protease | High | <5% |
| Prolyve PAC CONC | Acid protease | High | <5% |
| Extralyve LA2X | Xylanase | High | <5% |
| Lypaine 48 000 | Neutral protease | High | <5% |
| Flavour Seb NP | Neutral protease | High | <5% |
| Enz103 | | | |
| Prolyve exo DB 1000 | Protease | High | <5% |

1.-13. (canceled)

14. A protein ingredient, wherein said protein ingredient is a protein isolate or a protein concentrate, wherein said protein ingredient is in a dry form and wherein said protein ingredient has at least one of the following properties:
said protein ingredient comprises at least 90% of particles having a diameter greater than 100 μm,
said protein ingredient has an aerated density greater than 0.5 g·cm⁻³,
said protein ingredient has a tapped density greater than 0.6 g·cm⁻³,
said protein ingredient has a low convexity, a low elongation and a homogeneous circularity,
said protein ingredient is flowable,
said protein ingredient is dispersible and/or
said protein ingredient is wettable.

15. The protein ingredient according to claim 14, obtained by a process comprising a step of compressing a protein isolate or a protein concentrate followed by a step of milling.

16. A composition comprising at least one protein ingredient according to claim 14.

17. The composition according to claim 16, wherein said composition is a food composition, a feed composition, a pet-food composition, a cosmetic composition, a nutraceutical composition or a pharmaceutical composition.

18. A method for producing a protein ingredient according to claim 14, wherein said method comprises:
a) providing a protein concentrate or a protein isolate in the form of a wet powder,
b) compressing the wet powder of step a), to obtain pellets,
c) milling the pellets obtained in step b), to obtain a powder, and
d) classifying the powder obtained in step c), to obtain at least one target coarse fraction, optionally a fine fraction and optionally a coarse fraction having a size greater than those of the target coarse fraction(s).

19. The method according to claim 18, wherein step b) comprises compressing both the wet powder of step a) and the fine fraction obtained in step d).

20. The method according to claim 18, wherein step d) comprises classifying the powder obtained in step c), to obtain a fine fraction, at least one target coarse fraction and a coarse fraction having a size greater than those of the target coarse fraction(s) and wherein step c) comprises milling both the pellets obtained in step b) and said coarse fraction having a size greater than those of the target coarse fraction(s), to obtain a powder.

21. The method according to claim 18, wherein classifying comprises sieving and the target coarse fraction is obtained after at least one sieving comprised from 100 μm to 2000 μm.

22. The method according to claim 18, wherein said method is carried out in a continuous mode.

23. The method according to claim 22, wherein said method comprises:
a) providing a protein concentrate or a protein isolate in the form of a wet powder,
b) compressing both the wet powder of step a) and the fine fraction obtained in step d), to obtain pellets,
c) milling the pellets obtained in step b) and, optionally, the coarse fraction obtained in step d) which has a size greater than those of the target coarse fraction(s), to obtain a powder, and
d) classifying the powder obtained in step c), to obtain a fine fraction, at least one target coarse fraction, and, optionally, a coarse fraction having a size greater than those of the target coarse fraction(s).

24. A method for improving at least one handling property of a protein isolate or of a protein concentrate, wherein said method comprises a step of compression followed by step of milling.

25. A system for carrying out the method according to claim 18, wherein said system comprises:
optionally, at least one mixer,
at least one mechanical press,
at least one mill, and
at least one classification device,
wherein, when present, said mixer is connected to said mechanical press,
wherein said mechanical press is connected to said mill and
wherein said mill is connected to said classification device.

26. The system according to claim 25, wherein said classification device is connected to said mechanical press and to said mill.

27. The protein ingredient according to claim 14, wherein said protein ingredient is a plant protein ingredient or an enzyme composition.

28. The method according to claim 18, wherein said protein ingredient is a plant protein ingredient or an enzyme composition.
